(19) United States
(12) Patent Application Publication
(10) Pub. No.: US 2026/0020978 A1
(43) Pub. Date: Jan. 22, 2026

(54) SYSTEM ADAPTED FOR PROVIDING A THERMAL, MEDICAL AND THERAPEUTICAL EFFECT AT A SPECIFIC POINT OF BODY OF A PATIENT

(71) Applicant: CRYOTHERMO 4.0 SÀRL, Courroux (CH)

(72) Inventor: Marco VON GUNTEN, Courtedoux (CH)

(73) Assignee: CRYOTHERMO 4.0 SÀRL, Courroux (CH)

(21) Appl. No.: 18/868,092

(22) PCT Filed: May 24, 2023
(86) PCT No.: PCT/IB2023/055335

§ 371 Date: Nov. 21, 2024

(30) Foreign Application Priority Data
May 24, 2022 (EP) 22175140.7

(51) Int. Cl. A61F 7/00

(52) U.S. Cl. CPC A61F 7/0085; A61F 2007/0054

(57) ABSTRACT

The present invention relates to a system (1) adapted for providing a thermal and therapeutical effect to a patient (10) at a specific point of his body. The system comprises an element of contact (20a, 20b), adapted to be placed in contact to the point of body. The element of contact comprises a structural element (21) defining a tridimensionality shape. The system also comprises a thermal regulation device (50) and a regulation fluid circulating through the thermal regulation device and the element of contact. The present invention also relates to a process adapted to manufacture such a structural element.

### TECHNICAL DOMAIN

The present invention concerns a device such as a thermal device being used or usable as a compress, in particular a thermal compress. The present device is adapted to be placed on the body of a user or a patient, preferably at a specific location on the body such as a muscle, ligament, tendon or an articulation like a knee, a shoulder or a elbow. The present device is made of, or comprises, a material adapted to deliver a predetermined temperature toward such a specific point of the body. Such predetermined temperature refers to a hot temperature or a cold temperature or a cyclic temperature. The device can comprise additional medical and therapeutic such as electrostimulation and/or infrared irradiations as well medicine substances under form cream, liquid or gel.

### RELATED ART

Thermal compresses are already known. They are usually flexible pieces comprising a thermal gel, which are put on a part of the body where a muscular or articulation pain is sensed, or to improve physical recovery after an effort. They are often used by sportive people, or people having back pain or articular pain. Such thermal compresses need to be placed in an oven/micorowaves or in a cold storage, such as a fridge or a freezer, prior to be used on the body. This requires some time before the patient can benefit from its warm or cold effect. In addition, the effect of the known thermal compresses, once placed on the body, is limited over time, since its temperature equilibrates with the environmental temperature. Thus, the thermal benefit is unregular and may be disappointing. In addition, the compress can be too hot or too cold when placed on the body, resulting in a discomfort.

Another drawback of the known thermal compresses is that their shape is often not adapted to the portion of the body on which they are placed. The patient often needs to maintain it with his own hand, or to remain at rest, which limits his movements and activities.

The document US20190045857 discloses a garment adapted to regulate the temperature of the body based on a fabric comprising a branched tube network, in which a warm or cooled fluid can flow. Such a garment is provided for comfort.

The document US20180193186 discloses a thermal pad provided with fluidic channels.

The document US20150320591 discloses a in-ear thermal simulation system comprising an ear piece, wherein flows a thermal fluid, which is adapted to be inserted in the ear channel.

The document US20130238024 discloses a pad for thermal therapy based on a gas flow.

The document US20180098903 discloses a heat exchanger module adapted to be applied on a body part.

There is thus room to improve the known thermal compresses from a thermal, therapeutic, medical and morphological point of view.

### SHORT DISCLOSURE OF THE INVENTION

An aim of the present invention is the provision of a system or arrangement that overcomes the shortcomings and limitations of the state of the art. It is in particular an aim to provide a thermal arrangement with a longer lasting effect than a traditional compress, a more regular and constant temperature, or more adapted temperature, or a variable temperature or a combination thereof.

Another aim of the invention is to provide a system or an arrangement, in particular based on thermal exchanges, adapted to the morphology of the user or the patient at a specific point of body. This provides the advantage, between others, that the patient remains free of his movements, or is at least not forced to inactivity.

Another aim is to provide a method to produce a system or an arrangement, in particular based on thermal exchanges. It is in particular an aim to provide a fast and convenient process allowing to produce such a system or part of it on demand.

A further aim of the present invention is to provide a system allowing to deliver or apply on a given body part several different stimuli and/or therapeutic effects including thermal stimuli, in order to benefit from their synergistic effect.

A further aim of the present invention is to provide a system or an arrangement adapted to provide a mechanical input to a patient at a given part of his or her body, in particular a mechanical support and/or a mechanical positioning at a given body part. It is furthermore an aim of the present invention to combine such a mechanical input with a non-mechanical input such as a thermal stimulus, and/or a local medical or therapeutic treatment.

Additional advantages will become apparent through the detailed description below.

According to the invention, these aims are attained by subject-matter of the independent claims, object of the present invention. The details are object of the dependent claims.

The system or the arrangement according to the present description comprises an element of contact which can be used as a compress, and which allows thermal exchanges, in particular controlled thermal exchanges with the body at a specific point of the body. The element of contact according to the present invention may have a three-dimensional shape complementary to the specific point of body of the patient, so that it can easily fit with the morphology of the patient.

It can for example comprise a rigid or semi-rigid structural element such as a casing, a beam assembly, a grid, or any convenient structural element, adapted to provide and maintain a suitable tridimensionality shape, so that comfort is increased. The structural element defines a hollow space or a skeleton or an armature which is also adapted to support additional elements such as internal pipes, electrical wires, reservoirs of substances to be released for topical applications having health benefits such as a cream, a gel or a liquid. The structural element has a surface of contact adapted to be in contact with the skin of a patient. The surface of contact can be provided, at least partially, with a soft material such as a fabric, soft polymer or any material allowing to improve the comfort of the patient and the thermal transfer at the specific point of the body. Alternatively, the structural element can be inserted in a pouch or a bag for a more comfortable application on the body. Such a pouch or bag may comprise a gel or be filled with bids of material adapted to transfer thermal energy. The structural element can be made of a rigid or semi-rigid material so as to maintain its tridimensionality shape.

The system or arrangement according to the present description can also comprises an external (dissociated) thermal regulation device, adapted to produce and maintain a predetermined constant temperature at the element of contact. The thermal regulation device can be connected to the element of contact by means of pipes so as to allow a fluid circulating within or along the structural element of the element of contact, while being regulated by the thermal regulation device.

The system or arrangement can be provided with one or more sensors, such as thermo-sensors, heart rate sensor, blood pressure sensor, skin temperature sensor, or other bio-sensors. In particular, the element of contact, more particularly, the surface of contact of the element of contact is preferably provided with at least one thermo-sensor.

The system or arrangement according to the present disclosure preferably comprises a control unit, adapted to activate or deactivate the circulation of one fluid or more than one fluid. The control unit can in addition control the temperature of such circulating fluids, at a predetermined value, or between several predetermined values, as well as the variation of such temperatures such as increase or decrease rate of temperature or cyclic variation of temperatures. In case several fluids are circulating, the temperature and temperature variation of each of these fluids can be independently controlled.

The system or arrangement according to the present description may be provided with one or a combination of several different additional features depending on the needs. For example, it can be provided with communication means so as to be connected to a remote electronic device and communicate data therewith, including control data to pilot the thermal regulation device and/or data sensed by one or more sensors.

The system or arrangement of the present description may in addition comprise one or several storage compartment or reservoirs, combined with one or several porous areas adapted to deliver some quantities of biologically active compounds or mixtures to a specific point of the body. By this way, a substance can be delivered to the skin in contact with the element of contact.

The thermal regulation device comprises one or both heating device and/or cooling device so that the requested temperature can be produced. It further comprises the necessary elements such as pump and compressor, as well as all necessary features adapted to provide heat or cold. It can comprise at least one cold generation device such as Peltier device or chemical Peltier effect for cooling a circulating fluid. In addition, or alternatively, it can comprise at least one warm generation device such as Peltier devices, resistors, etc for warming a circulating fluid. A control unit is adapted to maintain the temperature at a predetermined constant value, to increase or decrease the temperature according to a predetermined rate, or to vary the temperature according to predetermined cycles.

The system or arrangement according to the present description may in addition comprise one or several tracking means adapted to determine and follow the status of the product (compress) including the structural element 21 or of the application (usage), including the medical or therapeutic usage and/or the monitoring of the corresponding data Tracking means can take the form of a RFID or any other suitable device.

The structural element of the element of contact may be sufficiently rigid so as to be used as a fixing element, for example in case of broken bones. The present system or arrangement can thus be used as a splint, a plaster cast, or in combination with such a splint or plater cast. The present system or arrangement can also be used for examples as a knee pad, or an elbow pad, or in combination with such a knee pad or elbow pad.

The present system or arrangement may be used by any individual either at home or in the framework of medical environment such as an hospital, a physiotherapeutic office. It can be used either alone or in combination with other treatment or medical devices. As an example, the present system or arrangement can be used by a sport practitioner or professional workers after an intense physical activity so as to improve physical recovery or to relieve potential muscular, ligament, tendon pain or articular damage. It can be used alternatively before an intense physical activity, so as to prepare the body or part of the body to such an intense effort.

In the present disclosure, the term “specific point of the body” denotes any part of the body in necessity of attention or treatment and/or stimulus and/or therapy. It can be a point of injury such as a body pain as luxation, a local inflammation, rheumatism, osteoarthritis, a sprain, a hernia or a bone fracture. It can alternatively be a point without declared injury or pain, but in need of preparation before an effort or in need of protection during an effort. As such, a specific point of body can denote an articulation such as a knee, an elbow, a wrist, an ankle, a heel or a shoulder. It can also denote a muscle such as the knurl, a thigh or a bicep. Such a specific point of the body can be characterized by its three dimensional shape, which is specific from the given point of body. The tri-dimensional shape can also be specific from the patient's morphology. A specific point of body preferably denotes an external part of the body, such as arms, legs, neck. It thus preferably excludes any internal areas such as mouth, nostrils, ear as well as any other body orifices.

In the present disclosure a “patient” denotes any person using the present system or arrangement to benefit from thermal and/or therapeutical effects and/or any necessary stimuli at a specific point of his body. A patient may denote a person having an injury, a local inflammation or any pain necessitating a thermal treatment. A patient also denote any person having no specific pain but willing to prepare for a physical activity such as a sportive competition or a hard physical work. A patient can also denote a user aiming at a relaxing time with a thermal effect or any related stimuli of the present system or arrangement.

The terms “stimuli”, “stimulus” and related terms denote any signal, effect or substances delivered to the patient at one or several specific points of body. A stimulus include a thermal effect as here described, either cold or warm or of variable temperature, along a given period of time. A stimulus, and in particular an “additional stimulus” denotes the delivering of one or several signals such as an electrical signal, a magnetic signal, light irradiation, ultrasounds, mechanical pressure or any related signal. Alternatively or in addition, a stimulus, and in particular an “additional stimulus” denotes the releasing of a substance or a composition such as a gel, a liquid, a cryo-gel, a cryo-gas and any related product at the point of body in need thereof. Such a substance or composition can comprise, or be free of, pharmaceutically active substances, comprise or be free of, well-being substances. Such releases substances or compositions are adapted for topical application. A stimulus can be adapted to provide comfort or well-being to a patient, including muscular reinforcement, sport preparation, retrieving after sport, action against fatigue and related actions. The intensity of the corresponding stimulus or the dosage and composition of the corresponding substance is adapted accordingly. Alternatively, the stimulus can be adapted to provide a therapeutic or medical treatment to the patient at the specific point of body. Examples of such therapeutic or medical treatment includes anti-inflammatory treatment, arthritis treatment, rheumatoid arthritis and pain.

With respect to what is known in the art, the invention provides the advantage of a more convenient and a better controlled device based on thermal exchanges and/or alternative stimuli.

### SHORT DESCRIPTION OF THE DRAWINGS

Exemplar embodiments of the invention are disclosed in the description and illustrated by the following figures:

FIG. 1: Overview of the arrangement according to one example of the present disclosure

FIG. 2A: Details of a compress according to an embodiment of the present disclosure

FIG. 2B: Details of a compress according to another embodiment of the present disclosure

FIG. 3: Transversal view of a compress according to an embodiment of the present disclosure

FIG. 4: Thermal regulation device view according to an embodiment of the present invention

FIG. 5: Details thermal regulation device according to an embodiment of the present disclosure.

FIGS. 6A, 6B, 6C: Details of the elements of contact according to embodiments of the present disclosure.

FIG. 7a, 7b: Cross sectional view of a structural element according to examples of the present disclosure.

FIG. 7c: Face view of an internal pouch according to an example of the present disclosure.

FIG. 8: Cross sectional view of a structural element according to an example of the present disclosure.

FIG. 9: Details of a pouch comprising an internal liquid circuit according to an embodiment of the present disclosure.

### EXAMPLES OF EMBODIMENTS OF THE PRESENT INVENTION

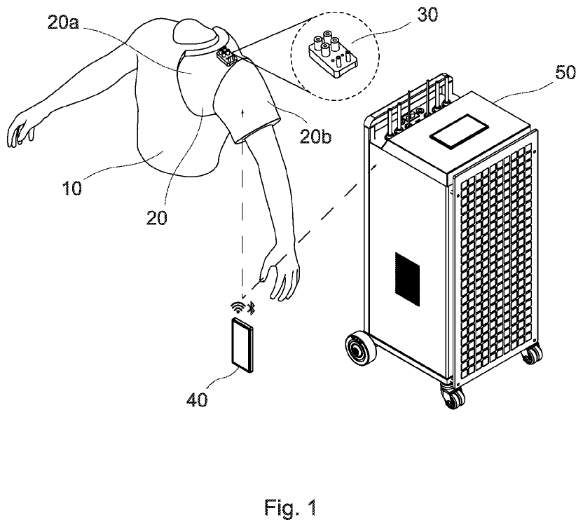

With reference to FIG. 1, the system 1 according to the present disclosure comprises one or more element of contact 20. Such an element of contact 20 is adapted to be placed in contact or close to a specific point of the body of a patient 10, preferably in direct contact with the skin. The element of contact 20 of the present system 1 is adapted to fit with the body of the patient, at a specific point of its body. This means that the surface of contact adopts or reflects the tridimensionality shape of the body at a specific point or area.

Fixation points and/or additional fixation element such as one or more belt, strap, a Velcro type element or clasp, buckle (not represented) can be arranged on the element of contact 20 to maintain it in close contact with the body. For example, a belt can be attached to opposite ends of the element of contact so as to be arranged around the abdomen of the patient 10. Depending on the specific point of body, such a belt can be used around a leg or an arm. Alternatively, or in addition, the shape of the element of contact is adapted so that the element of contact can remain on the body by itself. For example, the element of contact 20 can have a concave shape allowing to surround a member such as a leg or an arm in a way that it remains in contact with the body by itself. The element of contact 20 can alternatively or in addition comprise several subparts 20a, 20b. These subparts can be adapted to cooperate with each other. To this end, the subparts can exhibit complementary shapes adapted to combine them. Alternatively or in addition, arrangements such as snaps, belt, strap, a Velcro type element, clasp, buckle (not represented) can be envisaged. Alternatively, the subparts can be integral elements which are articulated so as to be adapted to the morphology of the patient. To this end they can be linked by means of a hinge, such as a flexible polymer or any other equivalent means. For example, it can comprise two half subparts each having a concave shape so that they can be arranged together around a member such as a leg or an arm, in a way to surround the corresponding member. The two half subparts 20a, 20b can be joined together by any suitable means or combination of means such as a snap, a belt, a strip, a clip and related means. Such an arrangement may also be used in case of a fracture of a bone, in particular of a long bone such as a femur, an ulna or any other long bone. The present element of contact may be used in replacement of the traditional plaster or resins used to maintain the bone fragments.

FIG. 1 shows an example of such an element of contact 20 having two subparts 20a and 20b, each one being adapted to, or complementary to the local shape of the body. For example, a first subpart 20a is adapted to fit with the top of the shoulder and a second subpart 20b is adapted to fit with top of the corresponding arm. The two subparts 20a, 20b are designed so as to fit one with the other so as to provide an element of contact covering all, or substantially all, the region of the body in need of attention. Although the two subparts 20a, 20b can be fixed together so as to provide globally one single rigid element of contact, they can also be arranged in a way to remain at least partly mobile with respect to one another. In the case of FIG. 1, the first subpart 20a covering the top of the shoulder and the second subpart 20b placed on the top of the corresponding arm may be joined by means of a flexible link such as an elastic so as to allow some moves of the arm independently from its shoulder. The connection may be rigid if necessary.

It is here understood that several subparts such as two, three, four or more can be used and assembled to cover the specific point of body in a more complete way. According to an embodiment, the present element of contact 20 can be designed as a modular assembly wherein each subpart has a predetermined shape adapted to cover a predetermined point of the body. The element of contact 20 is of course not necessary constituted by several subparts, as described here, but can consist in only one element.

An element of contact according to the present disclosure comprises at least one structural element 21 adapted to provide and maintain a predetermined tri-dimensional shape.

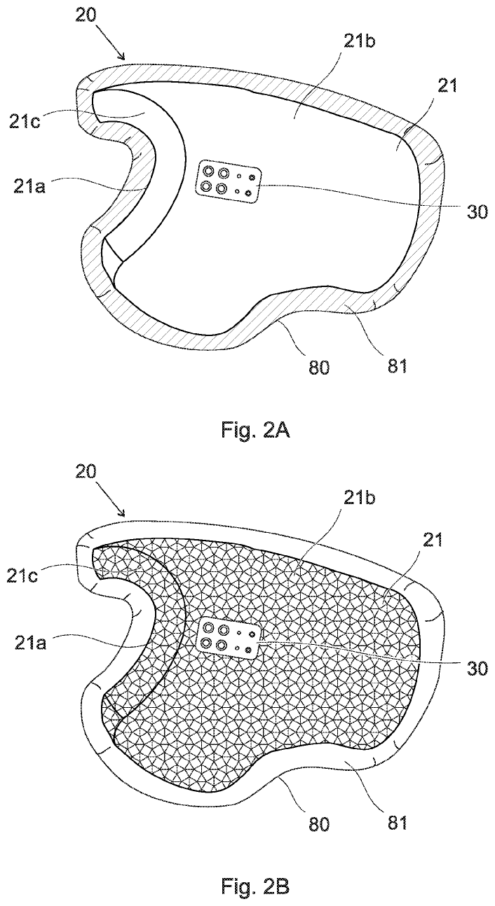

FIGS. 2A and 2B show an example of a structural element 21 according to an embodiment of the present disclosure. The structural element takes here the form of a casing 21C. It comprises a surface of contact 21a adapted to be oriented toward the body of the patient. The surface of contact 21a may be concave so as to surround a body part having a convex shape, such as a leg, an arm or any other convex part of the body. However, depending on the body part to be considered, the surface of contact 21a can have any adapted tri-dimensional shape. For example, it can be flat or substantially flat, or even convex, to be applied for example on the back or behind the knee.

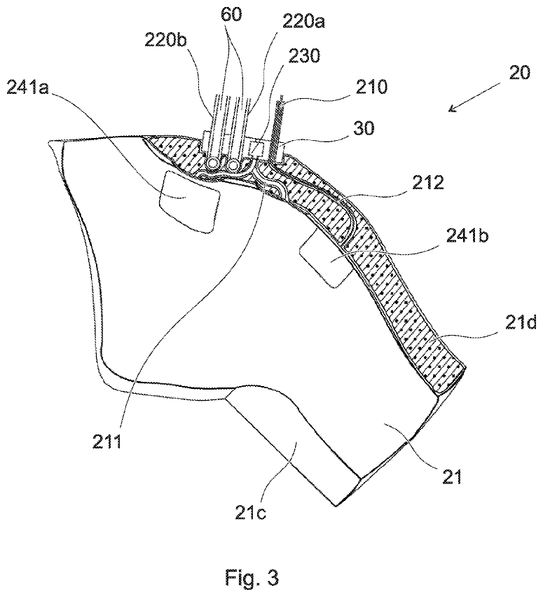

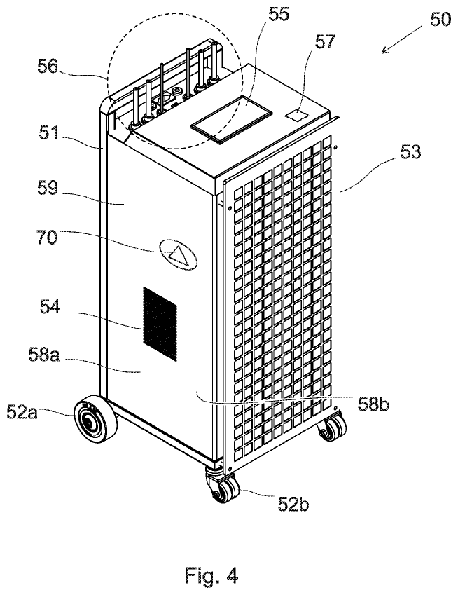

The structural element comprises a top surface 21b, opposite the surface of contact 21a. The top surface 21b is thus not assumed to be in contact with the patient, but to remain oriented outwardly when the element of contact 20 is placed on the body. The top surface 21b and the surface of contact 21a can be combined through an edge 21c so as to define an internal hollow space 21d (FIG. 3). According to an embodiment, the structural element 21 formed by the surface of contact 21a, the top surface 21b and the edge 21c is completely hermetic so that the internal hollow space 21d is waterproof. As such a liquid or a gel can be placed inside the structural element 21 of the element of contact 20 or can be circulated through dedicated connection points. Alternatively, the structural element 21 of the element of contact 20 is not waterproof as such but can comprise one or several waterproof internal pouch 25 placed inside the casing. Such an internal pouch 25 may be hermetic and filled with a liquid or a gel or any suitable material which can be used as thermal storage or thermal transfer. Alternatively, the internal hollow space 21d can comprise one or mor pipes wherein one or more fluid circulates. Beside the internal pouch or the pipes placed inside the structural element 21, the internal hollow space 21d may remain empty, meaning that the internal pouch 25, if any, is in direct contact with the internal surface of the structural element 21. Alternatively, the internal space 21d of the structural element 21 may be filled with a material 26 adapted to conduct thermal energy from the internal pouch 25, if any, or from the internal pipes, if any. Thus, a thermal inertia can be obtained for a better and/or longer thermal effect. The internal pouch 25 and/or the thermally conducting material 26, if present are preferably arranged in a way to maintain close contact with the tri-dimensional surface of contact 21a over its whole surface.

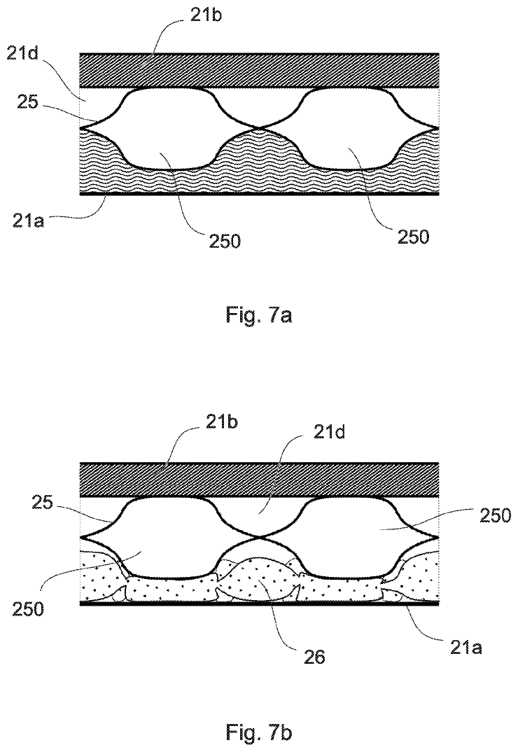

According to an embodiment better shown in FIGS. 7a and 7b, the internal pouch 25 comprises several cell 250 filed with a suitable gel or liquid. The cells 250 are embedded in a structure such as a polymer structure so that the pouch 25 remains in contact with the surface of contact 21c, whatever is its tri-dimensional shape and/or its orientation. In other words, the internal pouch 25 keeps its shape whether the casing is oriented vertically or horizontally. Alternatively or in addition, the internal pouch 25 can be glued on the surface of contact 21a, or fixed to said surface, or integrated into said surface, as shown in FIG. 7a. FIG. 7b shows an example wherein a thermally conducting material 26 is arranged between the internal pouch and the surface of contact 21a. FIGS. 7a and 7b are not intended to limit the scope of the present invention to this specific arrangement. For example, the cells 250 can be spaced apport from each other, or on the contrary arranged on several different layers withing the corresponding structure. Alternatively, several different individual layers of cells 250 can be arranged in contact to each other. Alternatively, a thermally conducting material 26 can be arranged between two distinct layers of cells 250. The structure in which are embedded the cells 250 can further comprise stiffeners or reinforcing elements.

FIG. 7c shows a representation of an internal pouch 25 having several cells 250. Several pouches 25 can be arranged inside the structural element 21 side by side, or with partial overlap, and/or superimposed.

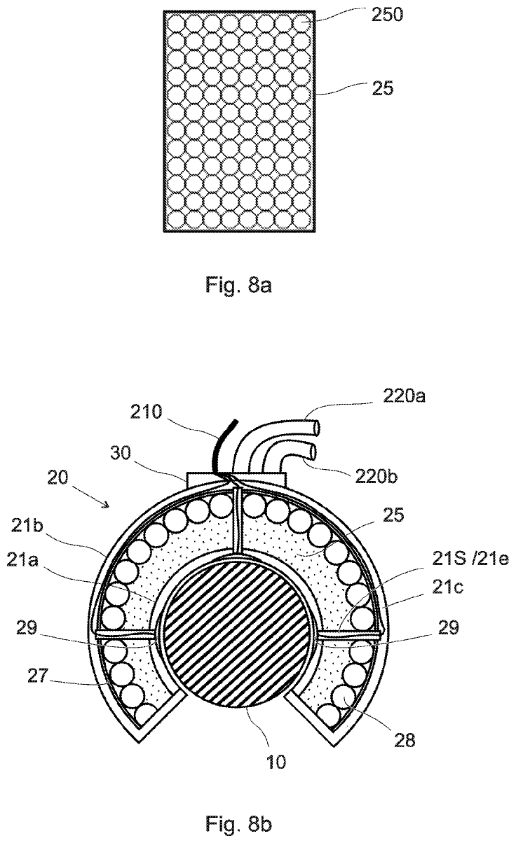

FIG. 8 shows an example wherein the internal pouch 25 or corresponding cells 250 are uniformly distributed inside the internal hollow space 21d of the structural element. Other non uniform repartitions can however be envisaged depending on the needs.

According to an embodiment, better shown in FIG. 8b, the structural element 21 can comprise internal reinforcing element 21e. For example, reinforcing elements can be connected or integral to the top surface 21b and opposite surface of contact 21a through the internal hollow space 21d. The reinforcing element can take any suitable form such as strait beams, arcuate ribs, honeycomb structure or any equivalent. The reinforcing element is preferably designed so as to be easily printed by means of an additive manufacturing process. The reinforcing element can further be hollow so that electrical cable can be inserted inside. Alternatively, reinforcing element can form pipes wherein a liquid can circulate. Alternatively or in addition, the reinforcing element is used to provide structural support to the internal pouch 25 and/or the corresponding cells 250.

According to another embodiment, one or more of the solid surfaces of the structural element, such as the surface of contact 21a, the top surface 21b or the edges 21c, is replaced by beams or ribs or grids, so as to lighten the structural element without prejudice to its tri-dimensional maintenance. As such, the structural element 21 can take the form of a skeleton 215.

The element of contact 20 further comprises at least one first fluid connection point 220a, 220b adapted to receive a fluid or several distinct fluids inside the structural element 21. According to an embodiment, a fluid can be input in the internal hollow space 21d of the structural element 21, either directly if the structural element is waterproof or through some internal pipes and/or internal pouch, so as to be stored inside the element of contact.

If the structural element 21 denotes a waterproof solid surface, so as to provide a casing, it can comprise some tubular recesses within the material of the envelop, allowing the circulation of the fluid. Alternatively, when the structural element is a skeleton 215, the fluid can be circulated through internal pipes. FIG. 8b provides an example wherein internal pipes 28 are distributed. In an embodiment, the internal pipes 28 are superimposed with the internal pouch 25 and/or thermally conducting material 26 so as to not be in contact with the surface of contact 21a. This provides the advantage of avoiding a too sudden or extreme temperature variation at the patient's skin. Thus the temperature can be smoothly regulated through the internal pouch 25 and/or thermally conducting material 26. In this specific example, the internal pipes are arranged on an external periphery of the structural element 21, while still being enclosed or inside the structural element. The internal pipes 28 can for example be arranged immediately bellow the top surface 21b or the corresponding coating or insulation layer 27. This does not exclude alternative arrangement wherein, the internal pipe 28 is place in a more central position inside the structural element 21.

The internal pipes 28 are preferably arranged so as to allow circulate a liquid from a single input to a single output, while covering most of the surface of the structural element 21. Tho this end, the internal pipe 28 is provided with the suitable circumvolutions. The internal pipe can be ducts of all kinds of hollow sections, including hollow cylindrical tubes, either rigid or flexible.

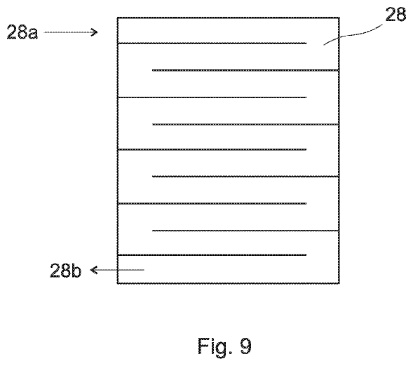

Alternatively, internal pipe 28 can take the form of a plate having internal hollow spaces adapted to guide a liquid from an inlet 28a to an outlet 28b (FIG. 9). Such a plate can be arranged in contact with the internal pouch 25 and/or thermally conductive material 26. The plate can be flexible so that it can easily be inserted in the structural element, independent of its tri-dimensional shape.

Close to the internal side of the surface of contact 21a, or even integrated to the surface of contact 21a, additional stimulating devices 29, including devices for medical and/or therapeutic treatment, can be arranged so as to provide the patient with different types of stimulations as needed. Such additional stimulating devices 29 can be selected among fluid reservoir, electrical patches or electrodes, light irradiating devices such as infrared emitting lights, vibrating devices, magnetic or electromagnetic devices, inflating devices adapted to locally increase the mechanical pressure, as well as any suitable arrangement. For example, the additional stimulating device 29 can denote a gel or a fluid reservoir, provided with a micropump and a porous membrane adapted to release a substance onto the patient skin. The micropump can be activated on demand or according to a predetermined program. Similarly, a flexible membrane can be filled with compressed air or another fluid so as to locally increase the pressure. A micropump can be arranged to this end. Different stimulating devices can be activated together so as to provide a synergistic effect. For example, a therapeutic gel can be released from a dedicated reservoir toward the patient skin, while a vibrating device and/or a light irradiation is activated. All these additional stimulating devices are concomitant to a temperature program, either warm or cool.

The additional stimulating devices 29 can be individually piloted from the regulation device 50. For example, a menu can be provide to allow selection of an action by the patient or another user.

According to an embodiment, the surface of contact 21a can be provided with sensing means such as surface temperature sensors, pH sensors, blood pressure sensors, or patches or probes. The sensing means can be localized at the additional stimulating device or nearby, or arranged at different locations on the surface of contact 21a.

Although the above-mentioned additional stimulating devices 29 are represented close to the surface of contact 21a in FIG. 8, other arrangements can be envisaged.

Although the sensing means are described here to sense the patient conditions, other sensing means can be dedicated to control the present system. For example, temperature sensors can be lodged inside the internal space of the structural element so as to sense the actual temperatures of the pipes or the surrounding environment, including the internal pouch 25 or thermally conducting material 26.

According to an embodiment, each one of the sensing means or some of them are connected to a computing means adapted to detect or diagnose a therapeutic condition or a physiological need. Such a computing means can further be adapted to provide a suitable stimulation program, based at least on a temperature application or variation, potentially combined with an additional stimulation through one or several of the additional stimulating devices 29. Such a computing means can be included into the data computing unit 40 described here. The corresponding stimulation or combined stimulations can thus be adapted for providing a suitable therapeutic treatment or be limited to comfort improvement.

According to an embodiment, the surface of contact 21a can be provided with a soft layer such as a polyurethan (PU) or thermoplastic polyurethan (TPU) or any equivalent so as to provide more comfort at the patient skin. In particular, the contact layer is defined so as to satisfy the medical regulation, for example to avoid any allergy and to allow dispensing a gel or a liquid.

According to an embodiment, one or both of the top surface 21b and the edge 21c can be provided with a soft layer for aesthetic or medical reasons. The soft layer covering the top surface and the edge may be identical or different from the one covering the surface of contact 21a.

According to an embodiment, the fluid can be circulated through the structural element 21 being a casing 21C or a skeleton 215 in a continuous way or in a semi continuous way. To this end, at least two first fluid connection points 220a, 220b are necessary. These first fluid connection points are each connected to a pipe, one being used as an input pipe and the other one being used as an output pipe. These pipes are connected to a regulation device 50 adapted to circulate the fluid through the pipes. The regulation device 50 comprises at least one pump 70, such as a peristaltic pump, or equivalent circulating means adapted to circulate the fluid. Although the flow rate can be fixed and determined by construction, the thermal regulation device 50 can comprise a means to vary, adjust or adapt the flow rate according to the needs. For example, the speed of the pump 70 can be controlled to provide a regulated flow rate of the fluid, either constant, or varying according to predetermined parameters. In a semi-continuous way, the liquid can be circulated through the structural element 21 being a casing 21C or a skeleton 215 for a given time and stopped for a given time. It can then circulate again without removing or connecting pipes, when the temperature reaches a predetermined threshold for example.

According to a specific embodiment, the first fluid connection point 220a, 220b can be disconnected from the input and output pipes once a fluid is placed inside the element of contact. In this case, the patient can benefit from the thermal effect while being free of any fluid connection with the external thermal regulation device 50 at least for a certain time.

According to an embodiment, internal surfaces of the structural element 21, or part of them, can be provided with a coating. In particular, an insulating coating or layer 27 can be provided on the internal side of the top surface 21b so as to avoid o limit the thermal losses. According to a preferred embodiment, the insulating layer is or comprises a reflecting layer adapted to reflect infrared irradiations toward the inside space of the structural element 21 and/or the patient's skin. Other surfaces can be provided with an insulating layer or coating depending on the needs.

The thermal regulation device 50 is further provided with at least one thermal regulation element (58a, 58b) adapted to regulate the temperature of one or several circulating fluids 60. Such a thermal regulation element preferably actively regulates the temperature of the fluid according to a predetermined program. It allows for example to maintain the temperature of the fluid at a predetermined value along a great time such as more than half an hour, more than an hour or even an indefinite time, until the thermal regulation device 50 or the corresponding thermal regulation element is stopped. The thermal regulation element can also be adapted to vary the temperature of one or several fluids according to a predetermined program. It can for example allow a continuous increase or a continuous decrease of the temperature between predetermine threshold values, with a predetermine temperature ramp. Alternatively, or in addition, the thermal regulation element can automatically adapt the temperature of the fluid according to one or more parameters values received from one or more sensors.

At least one thermal regulation element can be adapted to produce heat so as to warm a fluid above the body temperature or above the environment temperature. The temperature of the fluid can thus be maintained at a value comprised between 30° and 50°, preferably between 40° C. and 45° C. Alternatively, the at least one thermal regulation element can be adapted to produce cold so as to cold the fluid at a temperature lower than the body temperature or the environmental temperature. The temperature of the fluid can thus be decreased and maintained at a value comprised between −5° C. and 10° C., between −5° C. and 0° C., or between 0° C. and 5° C. or between 2° and 3° C. It is understood that alternative range of temperatures can also be selected according to the needs. According to an embodiment, the above-mentioned temperature corresponds to the temperature applied to the skin of the patient.

The thermal regulation device 50 may comprise one of such thermal regulation elements, either for the cold or for the hot production. Preferably, the thermal regulation device 50 comprises two thermal regulation elements, one being a heat production element 58a, dedicated to warm the corresponding regulating fluid and the other one being a cooling element 58b dedicated to cooling the corresponding regulating fluid. In that specific case, two distinct fluid circuits may be provided each one having its own circulating fluid and its own second fluid connection points 564a, 564b, 565a, 565b on the thermal regulation device 50. Alternatively, the regulation device 50 comprises only one thermal regulation element adapted to produce either cold or hot temperatures, so that only one circulating fluid circuit is necessary.

It is understood that the number of first fluid connection points 220a, 220b can be adapted accordingly so that a warm fluid or a cold fluid or a combination of several fluids can be circulated through the element of contact 20. It is also understood that in case of several subparts 20a, 20b, each one of the several subparts is preferably provided with at least one first fluid connection point.

The circulating fluid 60 denotes here any thermal regulating fluid such as a heat-transfer fluid or a coolant, either liquid or gaseous. It can be based on liquid comprising glycols or any other equivalent heat transfer liquid.

The thermal regulation device 50 can be provided with a battery or any suitable rechargeable power storage. It can also be used directly through an electrical plug 562. An additional power source can be optionally provided such as a solar panel 53. The regulation device 50 may be provided with an air-exchange system 54 adapted to circulate air through the thermal regulation device 50. Such an air-exchange system may comprise one or more fans, one or more grids and any suitable means to make air circulated.

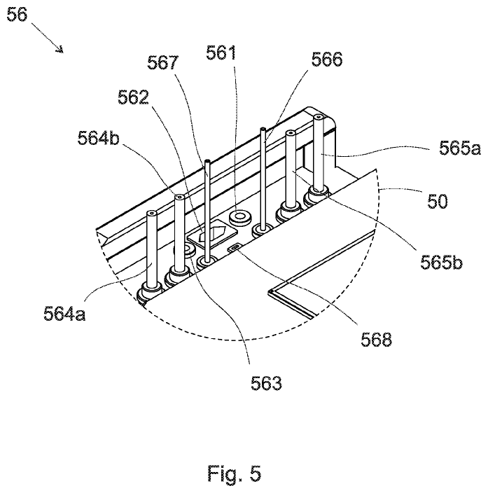

The thermal regulation device 50 can in addition be provided with an electrical stimulation system 59 adapted to deliver electrical stimuli to the patient through the element of contact 20. To this end, the regulation device 50 is provided with one or several electrical connection means 566, 567 (FIG. 5). The element of contact 20 is also provided with one or more suitable electrical connection means 210c, 210d (FIG. 3), and corresponding stimulation plates 241a, 241b, 241c, 241d or electrodes adapted to provide the electrical stimuli to the patient 10. The necessary electrical cables 212 can be lodged in the internal hollow space of the element of contact 20. The stimulation plates 241a, 241b, 241c, 241d are located on the surface of contact 21a so as to deliver the suitable stimuli to the body through the skin of the patient 10. Although electrical stimulation is mentioned here, it can be completed or replaced by any other necessary stimulations such as ultrasounds stimulation, magnetic stimulation, light irradiation such as infrared light irradiation. For example, the local infrared irradiation may be used in addition to a warm circulating fluid to irradiate some very specific points of the body in contact with the surface of contact 21a of the element of contact 20. A temperature different than the temperature of the circulating fluid can thus be obtained at precise dedicated points. The above-mentioned electrical stimulation system 59 may be used for medical and/or therapeutic treatment. Alternatively, they can be used for non-medical indications such as sport conditioning or retrieving or well-being.

The thermal regulation device 50 can be provided with a connection panel 56, wherein all the fluid connection points and the electrical connection means are localized. The connection panel 56 can comprise any additional connection means that can be necessary. For example, it can comprise one or more 12V connection plug 561, one or more 24V connection plug 563. It can comprise one or several USB connection plug 568 or other type of data connection plugs such as an Ethernet connection plug. It can comprise a communication unit adapted to receive and/or send data through wi-fi or Bluetooth or any suitable communication protocol toward a remote device such as a smartphone or a computer. Furthermore, the regulation device 50 may be connected or connectable to a communication infrastructure such as 4G or 5G, etc. . . . infrastructure.

The thermal regulation device 50 can in addition be provided with a command panel 57 allowing the user imputing data such as control data. For example, the user can select a program directly from its smartphone apps or from the command panel in a menu, or manually define a temperature, or a temperature variation or any other parameter of use of the regulation device 50. The regulation device 50 may also be provided with a display 55 or any equivalent visualisation means. Alternatively, or in addition, the regulation device 50 is connected or connectable to an external command panel and/or an external display. Such an external command panel and/or display can take the form of any suitable computing device such as a personal computer, a tablet, a phone terminal. The command panel and the display cooperate with the dedicated command program, which may take the form of an application, either localized on the command panel or remotely localized on a server.

The thermal regulation device 50 may be mobile or portative so that the patient 10 can bring it with him when it moves. As such, it can be brought at belt or via a shoulder strap. Depending on the equipment provided on the regulation device 50, its weight can be more or less important. For better comfort, the regulation device 50 can be arranged on a mobile support 51 such as a frame provided with a handle. The mobile support 51 may be provided with some wheels, either one pair or two pairs of wheels 52a, 52b. Alternatively, the thermal regulation device 50 can be fix and dedicated to an internal use, such as an hospital.

The thermal regulation device 50 is connected or connectable to a data computing unit 40, adapted to receive some data from the regulation device 50 or from the element of contact 21a, for example via the casing connection panel 30. To this end, the element of contact 20 can be provided with one or several sensors such as thermal sensor, adapted to sense the temperature at the skin of the patient 10 or elsewhere it may be necessary. Additional sensors may be arranged on the patient or adapted to sense some physiological parameters of the patient 10, such as heart frequency, blood pressure, body temperature. The computing unit 40 may be used as a control unit allowing to pilot the regulation device 50 in addition to, or instead of the panel control 57.

The thermal regulation device 50 and/or the element of contact 20 can be provided with a tracking means such as an RFID label. Data related to the status of the regulation device 50 and/or the element of contact 20 can thus be retrieved and monitored.

According to an embodiment, the element of contact 20 is provided with one or several reservoirs 230 adapted to receive some substances such as cream, oil, liquid, gel. A reservoir 230 is for example lodged in the casing connection panel 30 or in the internal hollow space 21d of the structural element 21, being a casing 21C or a skeleton 215, or incorporated or supported by the structural element. The reservoir comprises a surface of contact with the skin of the patient 10 so that the substance it contains can be delivered to the skin of the patient 10. Such a surface of contact can take the form of a porous surface or permeable surface allowing the substance to diffuse outside the reservoir 230. The reservoir may be filled with such a substance from a dedicated closable aperture placed for example on top surface 21b of the element of contact.

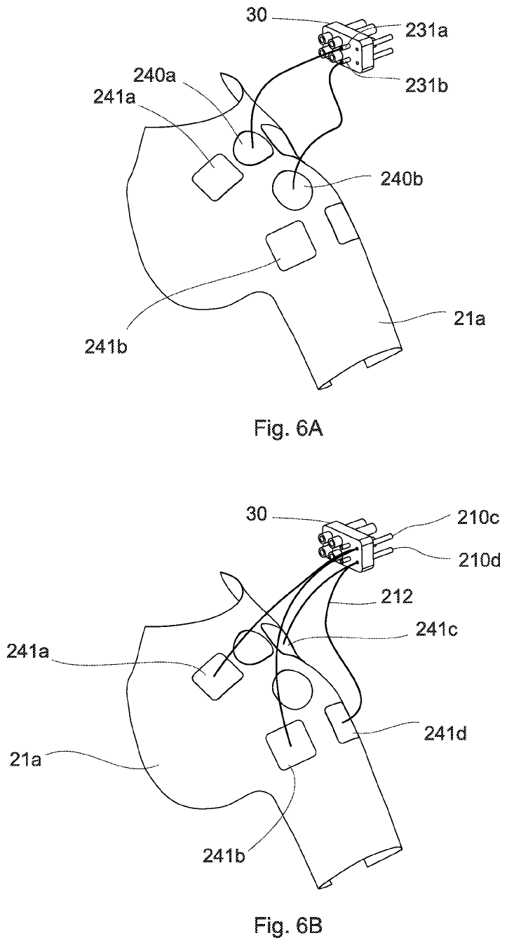

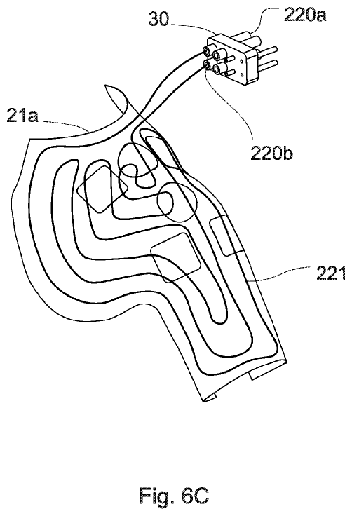

Referring back to FIGS. 1 to 3, the element of contact 20 can be provided with a casing connection panel 30, wherein all or most of the connection means are regrouped. The casing connection panel 30 can be included or embedded in the structural element of the element of contact 20. It is preferably arranged at a position easily accessible when the element of contact is placed on the body of the patient 10. It can be for example placed on the top surface 21b or on the edge 21c of the structural element. In case waterproofness is required, the necessary sealing is provided to join the casing connection panel 30 and the structural element 21 in a hermetic way. The casing connection panel 30 can thus comprises at least one first fluid connection point 220a, 220b. FIGS. 6A to 6C show examples of different connection modes related to the use of the element of contact 20, wherein only the surface of contact 21a is shown. The casing connection panel 30 comprises a first electrical connection means having two pins 210c, 210d. The corresponding electrical cables join these pins to some stimulation plate or electrodes 241a, 241b positioned on the surface of contact 21a. FIG. 6B shows an example wherein the casing connection panel 30 comprises a second electrical connection means comprising two pins 210c, 210d to connect stimulation plate or electrodes 214c, 241d. An ensemble of several electrical cables 211 joins the two pins 210c, 210d to a set of stimulating plates or electrodes 241a, 241b, 241c, 241d. A different stimulation than the stimulation provided through the first connection means can thus be operated. Alternatively, one of the first and second electrical connection means 566, 567 can be used to transmit some parameters sensed by sensors placed on the surface of contact 21a of the structural element 21, being a casing 21C or a skeleton 215. FIG. 6C shows an example wherein the casing connection panel 30 comprises a fluid connection point comprising two first fluid connection means 220a, 220b each adapted to receive a pipe, and an internal pipe 221 in contact or close to the surface of contact 21a of the structural element 21. The fluid delivered by the regulation device 50 can thus circulate through the element of contact and provide the requested thermal effect to the body of the patient 10. A second fluid connection point is also provided, while not connected to an internal pipe in the scheme. A second regulating fluid can thus circulate in the casing or within the structural element to provide a different thermal effect to the patient 10.

According to an embodiment, the casing connection panel 30 can be manufactured together with the structural element 21. Alternatively, it is manufactured separately and integrated or combined to the structural element 21.

According to another embodiment, the casing connection panel is adapted to connect individual connections means such as electrical, electronical, fluidic connection means and data exchange.

According to another embodiment, the casing connection panel comprises at least one combined connection means adapted to plug several types of connection selected from an electrical, an electronical, an air connection, a fluidic connection and data exchange.

In case more than on regulating fluid is used, they can be identical or different. One of the fluids can be well adapted for heat transfer while another one is well adapted for cooling effect. It is understood that the number and/or the type of connections is not limited and that several additional connection means can be envisaged depending on the needs.

Although the internal pipes 211 and electrical cables 212 are represented as being free, they can be embedded within the material of the structural element instead of crossing the internal hollow space 21d. This applicable when the structural element is a casing 21C or a skeleton 21S.

The patient 10 can be the user of the system 1 here described. He can easily install the element of contact 20 on a specific point of his body, connect at least one fluid connection point 220a, 220b with dedicated pipes connected to the thermal regulation device 50 and activate the thermal regulation device 50 to obtain the expected thermal effect. The patient 10 can select a pre-recorded program so that the thermal regulation device 50 provides an adapted thermal effect. According to an embodiment, once a suitable temperature is obtained, the patient can disconnect the fluid connection point from the corresponding pipes so that he is free to move remote from the thermal regulation device 50 while the thermal effect operates through the element of contact 20. According to another embodiment, the patient 10 can keep connected the fluid connection point so that an active thermal regulation is operated. The patient can select a program allowing to increase, decrease or maintain the temperature of the system 1, and more particularly of the element of contact 20. The patient can alternatively select a cyclic variation of the temperature. Alternatively, a patient can input some parameters to the thermal regulation device 50 according to specific needs. He can for example select a temperature to be maintained and controlled. He can select a lower and a high threshold of temperature, he can select an increase or a decrease rate of the temperature. He can select cyclic variations of temperatures including number of cycles and low and high temperature limits. Alternatively, the temperature can automatically vary or being adapted according to one or several values collected from one or more sensors. Such sensors include sensor for determining the temperature of the skin at the specific point of interest, the heart frequency, the blood pressure and any other suitable sensor or biosensor. According to a specific embodiment, several fluid regulations are circulating through the element of contact so has to provide different temperature at different specific points of the body. To this end, a given element of contact can be provided with several fluid connections so that several fluids can be connected. Alternatively, several elements of contact 20 are placed on the body at specific points of interest and each one of the elements of contact are connection to one thermal regulation device 50. The temperature can thus be independently adapted to the area. The patient 10 can in addition select one or more effect from an electrical stimulation, a substance release, an infrared stimulation, an ultrasound stimulation, a magnetic stimulation, according to pre-recorded programs.

Other users than the patient 10 may be involved in the operation of the present system 1. For example, a remote user such as a medical agent, a sportive coach, a data analyst, a researcher or any other authorized person can receive data from the system 1. Such data may relate to the parameters collected by means of dedicated sensors such as heart frequency sensors, body temperature sensor and/or blood pressure sensors. The data can be sent through a data computing unit 40 or any suitable device adapted to collect and/or process at least partly the data.

According to an embodiment, the structural element 21 is made, at least partially, preferably in its entirety, of a rigid material, so that its shape is predefined and cannot be changed anymore. The shape of the surface of contact 21a is thus adapted to fit with the portion of the body on which it is applied. In that case, the structural element can be made on demand, according to a fast and cheap process. For example, a rigid polymer can be processed through an additive manufacturing procedure so that it corresponds to the morphology of the patient. The additive manufacturing can be performed based on a numerical scan of the body to be considered, or any equivalent numerical recording means.

In practice, only the surface of contact 21a may be processed through an additive manufacturing method. For example, in case the structural element is a casing as described above, part of the casing 21C, comprising the edge 21c and the top surface 21b can be provided as a standard element, and based on this, the surface of contact 21a having the dedicated shape can be added. The process is thus fast and cheap, in a way that it limits the processing efforts. Before adding the surface of contact 21a to the rest of the casing 21C, a pouch and/or other suitable elements, as needed, can be arranged in the casing if necessary.

Alternatively, the whole casing 21C is made through an additive manufacturing operation on demand. This is more convenient in particular when very specific shapes need to be created.

In case the structural element is a skeleton 21S, all or part of the skeleton can be made through an additive manufacturing process.

The process described here with the example of a casing 21C having solid surfaces and a skeleton 21S, it can be operated with any suitable structural element, whether it comprises beams or ribs, or grids.

According to a specific embodiment, not only the structural element is produced by means of an additive manufacturing process. For example, internal pipes or internal circuits can also be manufactured with the same process and during the same time so as to improve the time of manufacturing. According to an embodiment, the internal pipes or circuits are organized in a way to form, as such, a structural element. According to another embodiment, internal pipes or circuits are combined or Integrated to a structural element. In case one or more reservoirs or pouches are considered, it can also be processed according to an additive manufacturing operation. A reservoir or a pouch can be made in the same time or through a distinct process step compare to the production of the structural element. In case porous or semi-permeable element are needed, they can be produced according to a suitable additive manufacturing process, using either the same component or different components.

Although additive manufacturing is preferred compared to other known processes, this does not fully exclude that moulding and/or machining some parts or all of the structural element can still be considered.

According to another embodiment, the structural element 21 is semi-rigid, meaning that it can be modelled by hand while keeping the shape one has urged it to adopt. The structural element 21 can therefore by made, at least partly, in soft polymer adapted to be modelled by hand. In this case, the structural element 21 can be modelled by the patient 10 himself in a way to be adjusted to his morphology at the specific point of his body. In some extend, the surface of contact 21a can be the only part that is semi-rigid while the remaining parts of the structural element 21 is rigid. Alternatively, the whole structural element 21 can be made of a semi-rigid material. The surface of contact 21a or the whole structural element 21 can take the suitable shape by pressing it at the specific point of body so that the surface of contact 21a takes the suitable complementary shape.

Rigid or semi-rigid structural element is applicable independently of whether it comprises only solid surfaces, and/or beams and/or ribs and/or grid.

The structural element 21 can be coated, at least on the surface of contact 21a, with one or several soft materials such as a silicon layer or a rubber layer or any suitable soft material. This is particularly suitable in case the structural element is directly applied on the body.

In a preferred arrangement, the structural element, or a combination of several structural elements, is included or inserted into an external pouch 80. Such an external pouch 80 is preferably used to improve the comfort of use of the element of contact 20. The external pouch 80 can be made for example of a fabric, of a textile made of natural fibres or synthetic fibres or both, of a soft polymer or mixture of polymers, on a natural polymer such as rubber, or of any other suitable material.

In addition, the external pouch can comprise additional materials, used as a filler 81 such as an internal gel or internal bids of material surrounding the structural element 21. This additional internal material provides the necessary flexibility and contact sensation to the element of contact, which is thus well adapted. The additional internal material can be selected in addition to improve the storage or the release of thermal energy.

It is understood that all the features and arrangement described here can be used independently or combined where applicable.

The present disclosure provides also a process for the manufacture of a structural element 21, or an assembly of combined structural elements. The process comprises a step of determining or selecting at least one specific point of body. The patient 10 may himself determine the location of the pain or injury his suffers from, or the location which needs a thermal effect or any other stimulus. He may also determine himself the nature of his pain or injury and/or the corresponding treatment or stimulus. The nature can for example relate to an inflammation or aches following unusual physical efforts he is aware of, or to a sprain following a wrong move he has made. Alternatively, a professional practitioner such as a doctor or a medical specialist can diagnose the origin, the precise location and/or the nature of his pain.

The process further comprises a step of determining the tri-dimensional shape of the body at the specific point of interest. Although a mould such as plaster moulded may be considered, the tri-dimensional shape of the specific point of body is preferably obtained through a scanning operation, so as to obtain precise numerical data related to the shape of the body at this location.

Once the tri-dimensional shape is known, the process comprises the step of determining a suitable tri-dimensional shape for an element of contact 20. Determining the suitable shape includes not only determining the tri-dimensional shape of the surface of contact 21a, but also the necessity to design one or more subparts 20a, 20b. The tri-dimensional shape of the structural element can be deduced from the step of determining the tri-dimensional shape of the element of contact. Alternatively, the tri-dimensional shape of the structural element 21 can be directly determined from the tri-dimensional shape of the specific point of body. The process may also include a step of determining the nature of the structural element or the structural elements 21. The nature includes for example the selection of solid surface, or beams, or grids, or a combination thereof. It can thus be decided to use a casing 21C or a skeleton 21S or an alternative structural element. The nature also includes whether internal pipes or circuits are integrated, or fused or combined with the structural element, or on contrary separately designed. The nature includes the presence or not of one or several reservoirs and/or pouches. The nature also includes the type of material used for the structural element and whether it should be semi-rigid or rigid. Once the suitable tridimensionality shape and the nature of the structural element or the structural elements 21 is determined, the process includes a step of manufacturing the structural element or the structural elements 21, including those related to the different subparts 20a, 20b. The manufacturing preferably includes or consists of an additive manufacturing. An additive manufacturing also denotes 3D printing operations and related processes.

According to a specific embodiment, the thermal regulation device 50 can be provided with means of pressurisation. In particular, the pouch may be inflated with pressurized air.

Reference symbols in the figures

| | |
|---|---|
| 10 | Patient |
| 20 | Element of contact |
| 20a, 20b | Subparts of element of contact |
| 21 | Structural element |
| 21C | Casing |
| 21S | Skeleton |
| 21a | Surface of contact |
| 21b | Top surface |
| 21c | Edge |
| 21d | Internal hollow space |
| 21e | Reinforcing elements |
| 210 | Electrical connection means of element of |
| | contact |
| 231a, 231b | Fluid pins for cream, liquid, gel connections |
| 210c, 210d | Electrical pins for electro-stimuli connections |
| 211 | Internal pipes for cream, liquid, gel delivery |
| 212 | Electrical cables |
| 220a, 220b | First fluid connection points |
| 221, 28 | Internal pipes or circuits for hot or cold fluid |
| 230 | Reservoir |
| 240a, 240b | Porous areas |
| 241a, 241b, 241c, 241d | Stimulation plates |
| 25 | internal pouch |
| 250 | Cells of internal pouch |
| 26 | Thermally conducting material |
| 27 | Coating |
| 28 | Liquid circuit pouch |
| 28a | Regulation liquid entry (IN) |
| 28b | Regulation liquid exit (OUT) |
| 29 | Additional stimulating devices |
| 30 | Casing connection panel |
| 40 | Data computing unit |
| 50 | Regulation device |
| 51 | Support |
| 52a, 52b | Pairs of wheels |
| 53 | Solar panel |
| 54 | Air-exchange system |
| 55 | Display |
| 56 | Connection panel |
| 57 | Command panel |
| 58a, 58b | Thermal regulation elements |
| 59 | Electrical stimulation system |
| 562 | 230 V Electrical plug |
| 563 | 24 V plug |
| 564a, 564b, 565a, 565b | Second fluid connection point |
| 566, 567 | Electrical connection means of regulation |
| 568 | USB |
| 561 | 12 V plug |
| 60 | Circulating fluid |
| 70 | Pump |
| 80 | Pouch |
| 81 | Filler |

1. System adapted for providing a thermal effect at a specific external point of body of a patient comprising:
one or more element of contact, adapted to be placed in contact or close to said specific external point of body, said one or more element of contact comprising at least one first fluid connection point adapted to circulate a regulation fluid through said one or more element of contact,
a thermal regulation device, comprising at least one pump and at least one second fluid connection point,
at least one regulation fluid whose temperature can be regulated by said thermal regulation device,
one or several pipes, fluidly connecting said first fluid connection point to said second fluid connection point,
wherein the at least one regulation fluid can be circulated through the element of contact by means of said at least one pump, wherein said element of contact comprises a rigid or semi-rigid structural element comprising a surface of contact, a top surface and an edge, defining together an internal hollow space, said surface of contact having its own tri-dimensional shape which fits with the morphology of said patient at said specific external point of body, said rigid or semi-rigid structural element (21) comprises a casing (21C), a skeleton (21S) or a combination of both, and in that said internal hollow space comprises internal pouch (25) and/or thermally conducting material (26), arranged in a way to be maintained in close contact with the tri-dimensional surface of contact (21a) over its whole surface.

2-3. (canceled)

4. System according to claim 1, further comprising internal pipes adapted to circulate said at least one regulation fluid through said internal hollow space of said structural element, said internal pipes being combined or integrated to, or forming at least part of said structural element.

5. System according to claim 1, further comprising internal pipes adapted to circulate said at least one regulation fluid through said internal hollow space of said structural element, said internal pipes being combined or integrated to one or several of said internal pouches.

6. System according to claim 1, further comprising internal reinforcing elements, integrated to said rigid or semi-rigid structural element.

7. (canceled)

8. System according to claim 1, wherein said element of contact further comprises at least one additional stimulating device selected from one or more of an electrical stimulation device, a magnetic stimulation device, light emitting device and a fluid release device, etc., is arranged on the surface of contact or nearby.

9. (canceled)

10. System according to claim 8, said at least one additional stimulating device being piloted by said thermal regulation device so that several different type of stimulation can be provided concomitantly, adapted to provide one or several stimuli providing comfort, therapeutic treatment or a combination of comfort and therapeutic treatment to said patient.

11. (canceled)

12. System according to claim 1, wherein the system further comprises or is connectable to a data computing unit adapted to receive and compute data from at least one sensor selected among a thermo-sensor, heart frequency sensor, blood pressure sensor and pH sensor, etc. arranged on the surface of contact (21a) or in the internal hollow space (21d) of the structural element (21).

13. (canceled)

14. System according to claim 1, wherein said thermal regulation device comprises one or both of a heat production element in a range of temperature between 35° C. to 45° C. and/or a cooling element in a range of temperature between −5° C. to 5° C. and one or more cooling fluid circulating circuit and one or more warm fluid circulating circuit.

15. (canceled)

16. A thermal regulation device comprising one or both of a heat production element and a cooling element and at least one pump adapted to circulate a thermal regulation fluid, said thermal regulation device further comprising piloting means adapted to pilot one or more of additional stimulating devices such as an electrical stimulation device, a magnetic stimulation device, light emitting device and a fluid release device, etc. wherein said regulation device (50) comprises or is connectable to a data computing unit (40) adapted to receive and compute data from at least one sensor selected among a thermo-sensor, heart frequency sensor, blood pressure sensor and pH sensor and in that said thermoregulation device is adapted to apply pre-recorded programs among a menu and/or to adjust parameters in real time such as temperature, temperature ramp, thresholds values of temperatures, flowrate of the regulating liquid, additional electrical stimulation, additional ultrasound stimulation, additional magnetic stimulation, additional infrared irradiation, additional vibration, and additional local mechanical pressure, according to sensed data.

17-18. (canceled)

19. Process for the manufacture of structural element of an element of contact as described in claim 1, said structural element being rigid or a semi-rigid and comprising at least a surface of contact having a tri-dimensional shape complementary of the tri-dimensional shape of a specific external point of a patient's body, said process comprising the steps of:
determining the morphology of said patient by means of one or several numerical scan, in particular tri-dimensional scan,
determining the tri-dimensional shape of a structural element according to the morphology of said patient, and
manufacturing the structural element, so that the tri-dimensional shape of the surface of contact fits with the morphology of the patient comprising at least one additive manufacturing step such as a 3D printing step.

20-21. (canceled)

22. System according to claim 1, wherein said internal pouch comprises several cells filled with a gel or a liquid, said cells being embedded in a structure adapted to maintain them in contact with said surface of contact independently of its tri-dimensional shape and/or its orientation.

23. System according to claim 22, wherein said cells are spaced apport from each other or arranged in several layers.

24. System according to claim 1, said internal hollow space comprises comprising both internal pouch and thermally conducting material, said internal pouch comprising several cells and said thermally conducting material being arranged between said internal pouch and the surface of contact or said internal pouch comprising several cells arranged in different layers and said thermally conducting material being arranged between said layers.

25. System according to claim 1, wherein said internal hollow space comprises several of said internal pouches.

26. System according to claim 6, wherein said reinforcing element denotes a structural element adapted to maintain said internal pouch in close contact with the tri-dimensional surface of contact over its whole surface.
